(19) United States
(12) Patent Application Publication
(10) Pub. No.: US 2017/0028131 A1
(43) Pub. Date: Feb. 2, 2017

(54) PUSH-PULL MEDICATION CONTAINER ADAPTER CAP FOR ENTERAL SYRINGE FILLING SYSTEMS

(71) Applicants: Nicholas J Perazzo, Rosedale, MD (US); Robert A Rosen, Owings Mills, MD (US)

(72) Inventors: Nicholas J Perazzo, Rosedale, MD (US); Robert A Rosen, Owings Mills, MD (US)

(21) Appl. No.: 15/274,702

(22) Filed: Sep. 23, 2016

Related U.S. Application Data
(63) Continuation-in-part of application No. 13/788,849, filed on Mar. 7, 2013, now Pat. No. 9,466,088.

(51) Int. Cl. A61M 5/178; A61M 39/20; A61M 39/24

(52) U.S. Cl. CPC A61M 5/1782; A61M 39/24; A61M 39/20; A61M 2039/2486

(57) ABSTRACT

A medication container adapter that utilizes a push-pull valve for adapting medication containers for use with enteral syringes that are filled on semi-automatic and automatic filling machinery. The enteral syringes are filled when the medication container is in the inverted filling position. The push-pull valve comprises of a stationary portion and a poppet slidably inserted into the stationary portion. The poppet is an annular member having a central channel and a fluted axial outlet from the central channel. The push-pull valve can be configured to be pressed onto an existing medication container adapter or manufactured as an integrated part of the medication container adapter so that it can either be screwed onto or pressed into the medication container neck.

### CROSS-REFERENCE TO RELATED APPLICATIONS

The present application is a continuation-in-part of U.S. application Ser. No. 13/788,849 filed Mar. 7, 2013, and a continuation-in-part of PCT/US2015/013217 filed Jan. 28, 2015, both of which are incorporated herein by reference.

### BACKGROUND OF THE INVENTION

1. Field of the invention

The present invention relates generally to enteral syringe packaging equipment and more specifically to an adapter cap incorporating a push-pull valve optimized for use in a fully or partially automated system for preparing patient-specific doses of selected pharmaceutical liquid medication for administration by syringe.

2. Description of the Background

Oral syringes are used to dispense liquids into the mouth. Enteral syringes are used to dispense liquids into the gastro-intestinal tract through a tube. As shown in FIG. 1, oral syringes utilize a male tapered tip. Enteral syringes utilize a female tip as shown in FIGS. 2A & 2B.

Hospital pharmacists prefer to fill just one type of syringe because they typically do not know whether a prescription will ultimately be administered orally or enterally. Another issue of great concern relates to mistaken use of syringes. Syringes intended to be dispensed into the mouth or into an enteral tube can, inadvertently, though rarely, be dispensed into an intravenous (IV) patient port. Such mistakes have caused injury and death to patients. Both of these problems have recently been addressed by the Global Device Suppliers Association (GEDSA). This industry group has introduced new devices referred to as ENFIT™ which include enteral syringes. The ENFIT™ devices are not compatible with luer connections or any other type of small bore medical connectors. This ENFIT™ design prevents the misconnection of enteral syringes to the patient's tubing port. As a result of the GED5A organizations efforts, the International Standards Organization created ISO CD 803069-3 which specifies the safe design for an enteral feeding connection. The new ENFIT™ enteral syringes comply with ISO CD 80369-3, can be used to administer medication orally or enterally, and cannot easily be connected to an incorrect patient tubing port.

As shown in FIG. 1, oral syringes have a tapered tip which can be used with a medication container to syringe adapter with an elastomer valve for filling the syringe when the container is in the inverted position.

The tapered tip of the oral syringe (See FIG. 1) penetrates the elastomer valve which enables the syringe to extract the liquid from the medication container. This adapter arrangement is not suitable for filling enteral syringes as the enteral syringe has a female fitting on the tip and is not compatible with the elastomer valve adapter used for the oral syringe.

To address this problem, a new Push-Pull Valve Adapter (for which this patent application is based) was developed by the Inventors specifically for use with the female tip of the enteral syringe (refer to FIGS. 5 & 6).

Prior to filling the syringe, the valve is closed (FIG. 5). During the filling of the syringe, a mechanical actuator FIG. 5 (valve opener/closer), located at the fill station, opens the valve FIG. 6. When the syringe has been filled, the same mechanical actuator closes the valve.

The initial design of the enteral syringe tip caused excess liquid to accumulate in the tip of the syringe which caused unacceptable fill accuracy for the smaller size enteral syringes. A new design which incorporates a center nozzle within the syringe discharge port addressed this problem (FIG. 5).

The change in the design of the discharge port of the enteral syringe required a new design for the syringe to medication adapter valve. This design changes obviated the prior art. A new adapter needed to fill both the smaller enteral syringes that had the center nozzle port (FIG. 5) as well as the larger syringes which did not have the center nozzle port (FIG. 11) without using change parts.

Thus, as shown in FIG. 2, two versions of enteral syringes now exist. As shown in FIG. 2(A) larger enteral syringes (typically 5 cc and above) utilize an internally-threaded female tip and no center nozzle. As shown in FIG. 2(B) smaller enteral syringes (typically 0.5 ml-3 ml) utilize an internal tapered or slip-nozzle with an enteral threaded female tip and a center nozzle.

Automated filling systems have been developed by Baxa, Inc., For Health Technologies, Inc., Intelligent Hospital Systems, Applicant National Instruments Co. (see, for example, Applicant's U.S. Ser. No. 13/788,849 filed Mar. 7, 2013 and others for the automated filling of syringes). However, the degree of automation in the hospital pharmacy for the packaging of oral/enteral syringes is limited due to the wide array of different syringes to fill and different medicine containers to fill them from. Bulk medications are commonly provided in variously sized bottles or containers having threaded screw caps that must be removed and replaced with container-to-syringe adapter caps. Most any attempt at automated filling of any syringes requires a modification to standard manufacturer-supplied medicine containers. Semi-automated and automated filling of syringes requires that the medication container be in the inverted position while the syringe is being filled. It is also necessary that the medication container cap be open during the time that the syringe is being filled, and closed when the syringe has been filled to prevent leakage. Thus, the manufacturer-supplied screw-on caps must be removed and replaced by an adapter cap that allows the syringe to be connected to the medication container. To fill oral syringes it is known to use an adapter cap with an elastomeric valve that allow the syringe tip (FIG. 1) to penetrate the inverted container. The tapered tip of the oral syringe would penetrate the elastomer valve while the medicine container is held in an inverted position, enabling the syringe to extract the liquid from the medication container without leakage.

Unfortunately, the elastomeric seal-type adapter cap does not work with enteral syringes which are manufactured in a variety of sizes with differing plunger configurations (FIGS. 2A, 2B). The prior art adapter arrangement is not suitable for filling enteral syringes (FIGS. 2A, 2B) since the enteral syringe has a female fitting on the tip and is not compatible with the elastomer valve adapter used for the oral syringe.

Given the diversity of enteral syringes and medicines available, any semi-automated (or fully-automated) system will need sufficient dexterity to manipulate all the myriad prescription bottles containing the pharmaceuticals to be dispensed as well as variously sized enteral syringes, bringing them together in a controlled environment to quickly and accurately fill and label each syringe and to verify its work as it proceeds in order to avoid errors in the process. Existing adapter caps are incapable of use with enteral syringes that are being filled on an automated or semi-automated basis when the medication container is in the inverted position. Consequently, existing adapter caps do not address the needs of medical institutions desiring a semi-automatic or automatic enteral syringe filling system when the medication container is in the inverted position. The present invention was developed to fill this void.

### SUMMARY OF THE INVENTION

To address the need to fill enteral syringes, on a semi-automatic or automated basis, with the medicine container held in the inverted filling position, a push-pull adapter cap is herein disclosed. When filling the syringe the valve is open. After the syringe has been filled the valve is closed. The push-pull valve is open or closed by a mechanical actuator at the automated or semi-automated filling station.

The push-pull adapter cap disclosed herein, when used with an enteral syringe filling system, enables hospital pharmacists to simplify and streamline their task, increasing the number of prescriptions that can be filled in a day, and improving patient safety and care by minimizing medication errors and the consequences that ensue.

### BRIEF DESCRIPTION OF THE DRAWINGS

The objects, features, and advantages of the present invention will become apparent from the following detailed description of the preferred embodiments and certain modifications thereof when taken together with the accompanying drawings in which like numbers represent like items throughout and in which:

FIG. 1 is a perspective view of a conventional oral syringes with tapered tip.

FIG. 2(A) is a perspective view of a large-sized conventional enteral syringe with internally-threaded female tip.

FIG. 2(B) is a perspective view of a smaller-sized conventional enteral syringe with an internal tapered or slip-nozzle and a female tip.

FIG. 3 is a perspective exploded view of a push-pull adapter cap 10 for retrofit application to a standard manufacturer-supplied medication container cap such as a Baxa™ or equivalent valve-less medicine container adapter cap with opening.

FIG. 4 is a perspective view of the push-pull adapter cap 10 of FIG. 3 illustrating how it interfaces an automated filling system.

FIGS. 5-6 are sequential perspective views of the push-pull adapter cap 10 of FIGS. 3-4 illustrating how it interfaces a smaller-sized enteral syringe (with internal tapered slip-nozzle and enteral threaded female tip) while in the automated filling system.

FIG. 7 is a side view of the poppet 84 used in the push-pull adapter cap 10 of FIGS. 3-6.

FIG. 8 is an end cross-section of the poppet valve of FIG. 7.

FIG. 9 is a side cross-section of the press-on ring 60 used for attaching the push-pull adapter cap 10 of FIGS. 3-8 to a standard manufacturer-supplied medication container adapter cap.

FIG. 10 is a side cross-section of a modified press-in version used for attaching the push-pull adapter cap 10 inside the neck of a standard manufacturer-supplied medication container cap.

FIGS. 11-12 are sequential perspective views of the push-pull adapter cap 10 of FIGS. 3-6 that illustrate how it interfaces a larger-size enteral syringe lacking a center nozzle. FIGS. 6 and 12 illustrate the device's ability to adapt to both small syringes (with nozzle) and large syringes (w/o center nozzle).

FIG. 13 is a cross-section of the poppet valve of the present invention inserted into a large enteral syringe and illustrating the poppet valve circumferential seal surfaces on the large enteral syringe in accordance with the invention. FIG. 14 is a cross-section of the poppet valve of the present invention inserted into a small enteral syringe and illustrating the poppet valve circumferential seal surfaces on the small enteral syringe in accordance with the invention.

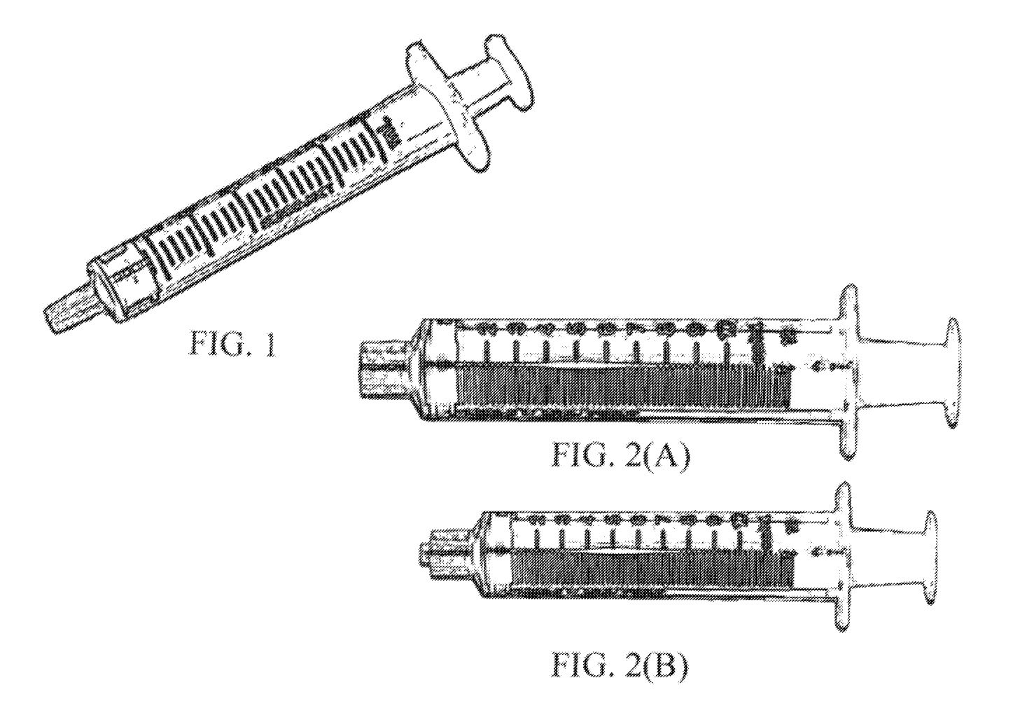

### DETAILED DESCRIPTION OF THE PREFERRED EMBODIMENTS

For the purposes of promoting an understanding of the principles of the invention, reference will now be made to the exemplary embodiment illustrated in the drawings and described below. The embodiment disclosed is not intended to be exhaustive or limit the invention to the precise form disclosed in the following detailed description. Rather, the embodiment is chosen and described so that others skilled in the art may utilize its teachings. It will be understood that no limitation of the scope of the invention is thereby intended. The invention includes any alterations and modifications in the illustrated device, the methods of operation, and further applications of the principles of the invention which would normally occur to one skilled in the art to which the invention relates.

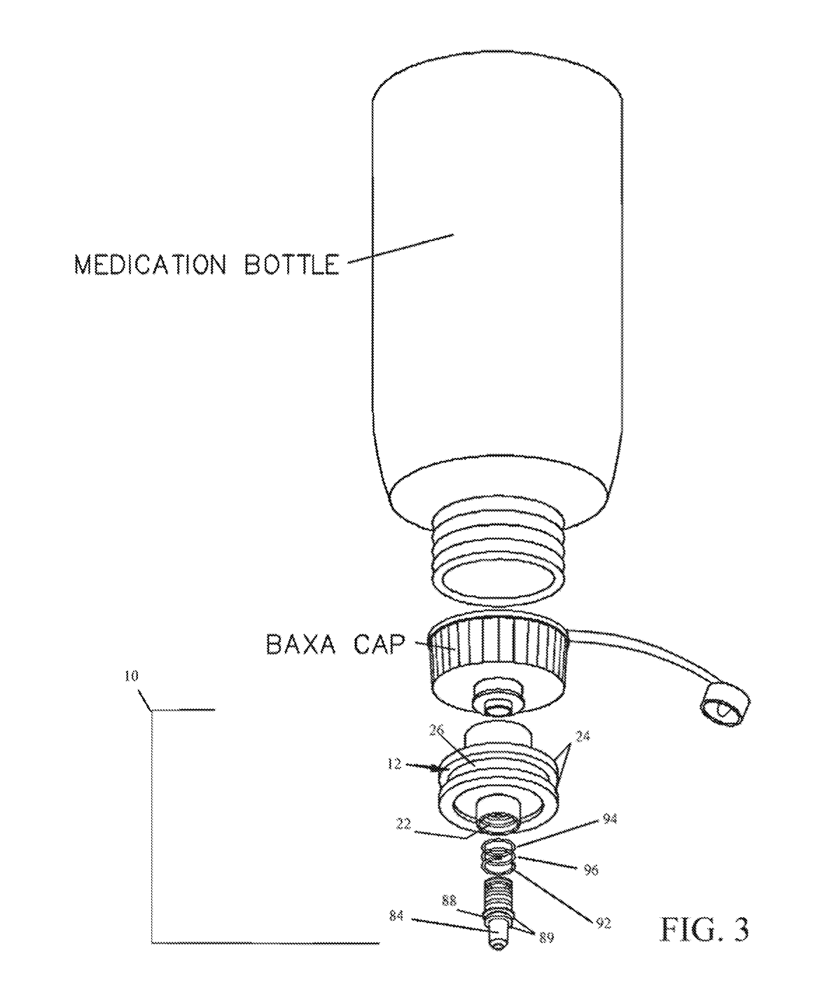

FIG. 3 is a perspective exploded view of a push-pull adapter cap 10 for retrofit application to a standard manufacturer-supplied (Baxa™ or Baxa equivalent) medicine container cap or equivalent valve-less medicine container cap with opening. For purposes of definition, this pre-existing medicine container cap is herein designated the “OEM cap.” Adapter cap 10 generally comprises a stationary portion 12 for attachment to the OEM cap, a poppet 84 slidably inserted into the stationary portion 12, a plurality of O-rings inclusive of a fluid seal 94 and two poppet seals 92, 96.

Stationary portion 12 comprises an annular body with a central passage 22 there through, the central passage 22 being defined by an inner wall having a specific progression of diametric variations. At one end of the body of stationary portion 12, where stationary portion 12 attaches to the top of the manufacturer-supplied medicine container cap, central passage 22 has a relatively large diameter sized to conform to and receive the nozzle of the manufacturer-supplied medicine container cap as described below with reference to FIG. 8. This forms a “press-fit” seal. It will be understood by one of ordinary skill in the art; however, that stationary portion 12 may be sized to fit other manufacturer-supplied caps by different means based on design preference, such as a screw-on connection.

Central passage 22 continues partially through stationary portion 12 at a relatively constant diameter but then constricts at a shoulder 98 to a smaller diameter sized to accommodate the elastomer poppet 84, as will be described further herein, which protrudes from the distal end of passage 22. The outer wall of stationary portion 12 further comprise two spaced annular flanges 24 separated by an annular groove 26 for alignment with a stationary yoke of the filling station, as further described herein. Similarly, the outer wall of poppet 84 is equipped with two spaced annular flanges 89 separated by an annular groove 88 for alignment with a movable yoke of the filling station, as further described herein.

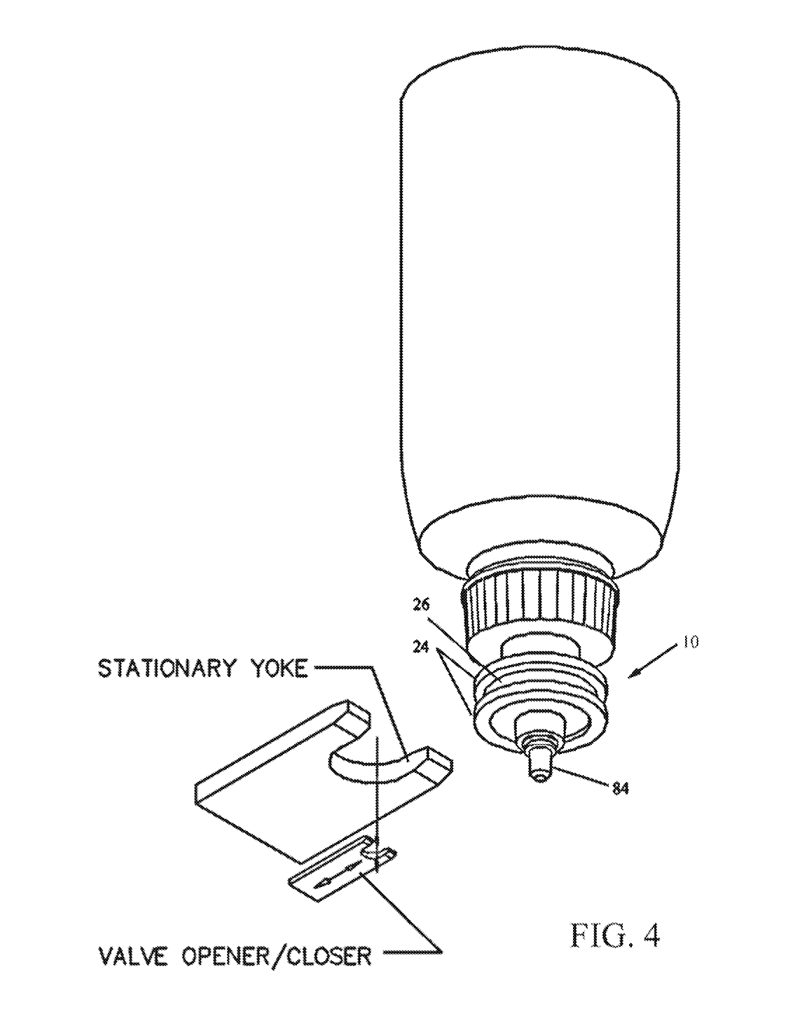

FIG. 4 is a perspective view of the push-pull adapter cap 10 of FIG. 3 illustrating how it interfaces an automated filling system. The automated filling system employs at least one anchoring yoke and at least one poppet-manipulating yoke, although one skilled in the art will recognize that counter-opposed pairs of such yokes may be used. The anchoring yoke(s) fit within the annular groove 26 of stationary portion 12 described above to thereby securely hold the medicine container by its manufacturer-supplied cap in a fixed position. The poppet-manipulating yoke fits within the annular groove 88 of poppet 84 described above to thereby insert and/or extract the poppet 84 from the stationary portion 12, thereby closing or opening the valve.

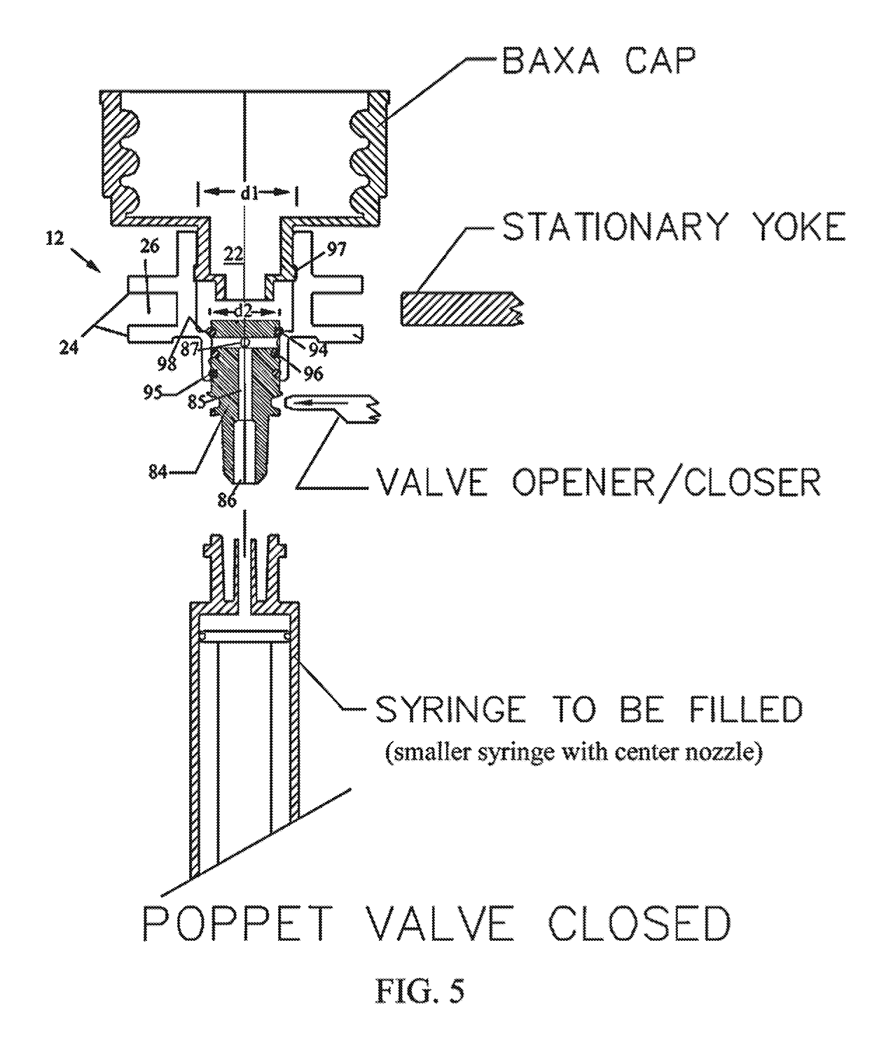

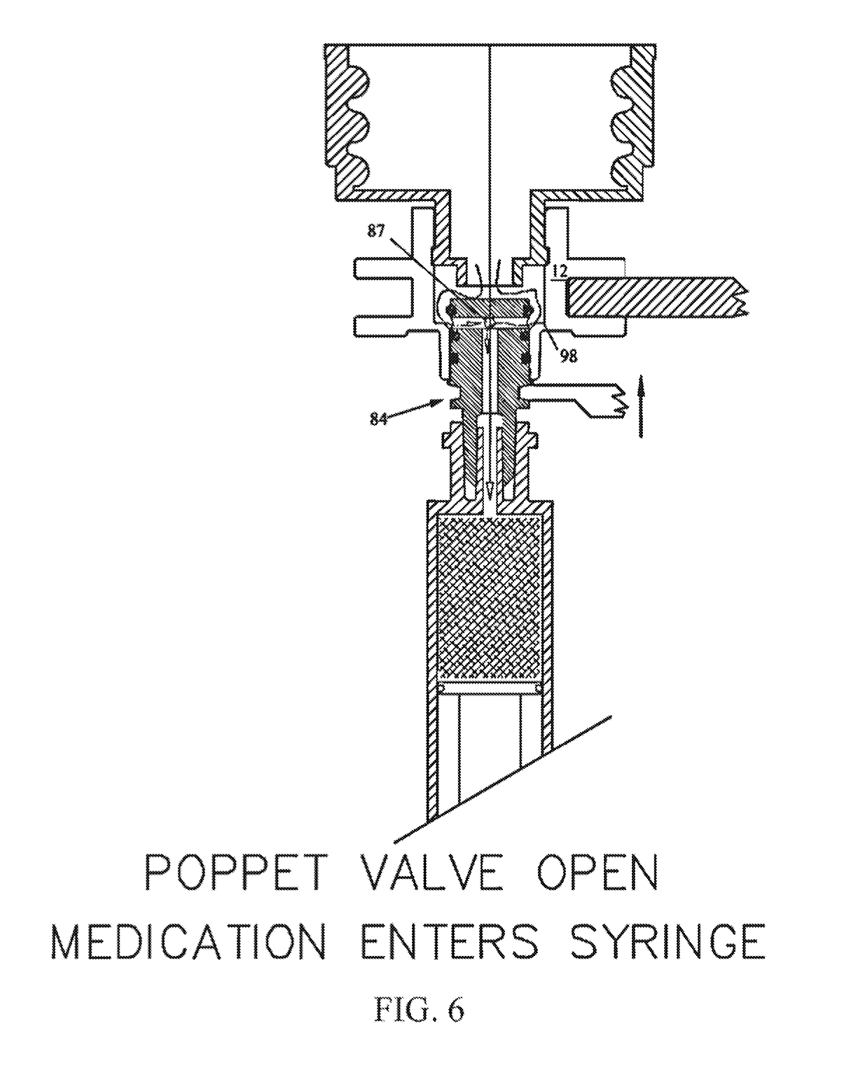

FIGS. 5-6 are sequential cross-sections of the push-pull adapter cap 10 of FIGS. 3-4 illustrating how it interfaces an enteral syringe while in the automated filling system. Note that the syringe in FIGS. 5-6 is a smaller-size enteral syringe with female tip and internal tapered slip-nozzle. In adapter cap 10 the central passage 22 continues approximately two-thirds through the stationary portion 12 at a relatively constant diameter d₁, to a point approximately even with the lower flange 26. However, that constant diameter passage is interrupted at about its midpoint by a shallow annular notch 97. Notch 97 provides for snap-fit capture of the OEM cap by its nozzle, which as shown is typically provided with a distal flange for snap-fit of a nozzle cap. The central passage 22 continues at constant diameter d₁ to a shoulder 98 where is constricts to a smaller diameter d₂ sized to accommodate the elastomer poppet 84, as will be described further herein, which protrudes from the distal end of passage 22. Poppet 84 likewise has a central passage 85 leading from a radial inlet 87 to an axial outlet 86. Note that the tip of poppet 84 is externally-tapered yet the central passage 85 is cylindrical leading to axial outlet 86. This way, the tip of poppet 84 exactly fits the moat between the enteral syringe female tip and internal tapered slip-nozzle, preventing any accumulation of medicine and undesired leakage.

In operation, when the poppet 84 is fully extracted as seen in FIG. 5 the inlet 87 is sealed within the smaller diameter d₂ portion of passage 22 and forms a fluid-tight seal. Conversely, when the poppet 84 is fully inserted as seen in FIG. 6 the inlet 87 pushes above shoulder 98 and is freed within the larger diameter d₁ portion of passage 22. Medicine is free to flow through the poppet 84 as seen by the arrows of FIG. 6 for filling of a syringe (below).

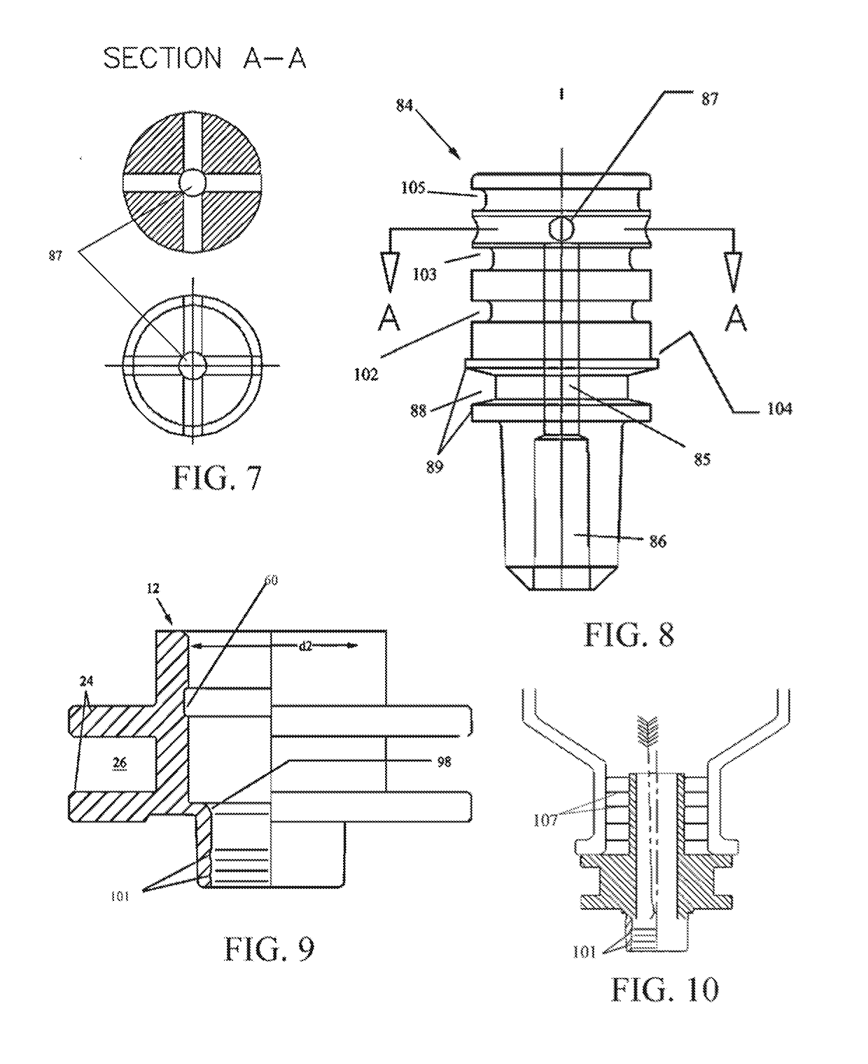

FIGS. 7 and 8 are a cross-section and side view of the poppet 84, which is an annular member formed with a plurality of grooves 101-105 spaced axially, and central passage 85 running centrally and axially and leading from a radial inlet 87 to an axial outlet 86. Radial inlet 87 may be any one or more inlet-passages extending radially into poppet 84 and in fluid communication with passage 85. Outlet 86 is enlarged and inwardly-fluted to receive and conform to the tapered nozzles of most enteral syringes. The grooves 101-105 include a lowermost groove 101 for receiving the smaller articulating yoke of the filling system which thereby inserts and/or extracts the poppet 84 from the stationary portion 12, thereby closing or opening the valve. In addition, three grooves 102, 103 and 105 are provided for three O-rings, inclusive of a groove 105 for a poppet seal 94 above the inlet 87 for sealing the poppet 84 against the shoulder 98 when fully open, and two lower grooves 103, 105 below the inlet 87 for fluid seals 95, 96 thereby preventing leakage out around poppet 84.

FIG. 9 is a side cross-section of the press-on ring 60 used for attaching the push-pull adapter cap 10 of FIGS. 3-6 to a standard manufacturer-supplied medication container cap. It is noteworthy that distal end of stationary portion 12 forms a nozzle through which the smaller diameter d₂ portion of passage 22 passes, and the inner walls of this nozzle are formed with two spaced grooves forming 0-ring seats 101 as shown for seating the fluid seals 95, 96 of poppet 84 when it is in its fully extracted position, and thereby ensuring a fluid-tight closure.

At the distal end of stationary portion 12 the outer diameter of the nozzle is sized to fit inside the female tip of an enteral syringe. The fluted outlet 86 of poppet 84 is configured to receive and conform to the tapered nozzles of most enteral syringes. This particular confirmation is well-suited for attachment to all variety of enteral syringes inclusive of a female tip with or without nozzles.

One skilled in the art will understand that other configurations may be used for attachment to other medicine containers. For example, the inner wall of the adapter cap along d2 may be defined by a simple inwardly-threaded connection for screw-insertion onto the threaded container neck. Alternately, the inner wall of the adapter cap 10 along d2 may be formed with a series of integrally formed inwardly-directed circular gripping ribs for gripping the neck of a medicine container by its threads.

FIG. 10 is a side cross-section of a modified press-in version used for attaching the push-pull adapter cap 10 inside the neck of a standard manufacturer-supplied medication container cap. Rather than fitting around the medicine container neck the ring fits inside, sealing by a plurality of resilient annular ribs 107 thereby ensuring a fluid-tight closure.

Alternatively, the ring may fit outside the medicine container neck using a plurality of inwardly-directed resilient annular ribs 107 to ensure a fluid-tight closure. In this case, as the neck of a medicine container is forced into the central void, the ribs 107 engage the threads on the outside of the neck of the bottle and flex slightly to permit the threads to pass. Once past, the ribs 107 spring back toward their original position and press against the neck to engage the threads and secure the adapter cap 10 to the container. Whether male or female, the flexure of the ribs 107 permits the adapter cap 10 to accommodate size variations in outside neck diameter and thread finish, and create a fluid-tight seal without the need for a specific thread pitch. The foregoing is set forth in more detail in co-pending application Ser. No. 13/788,849 filed Mar. 7, 2013, which is herein incorporated by reference in its entirety.

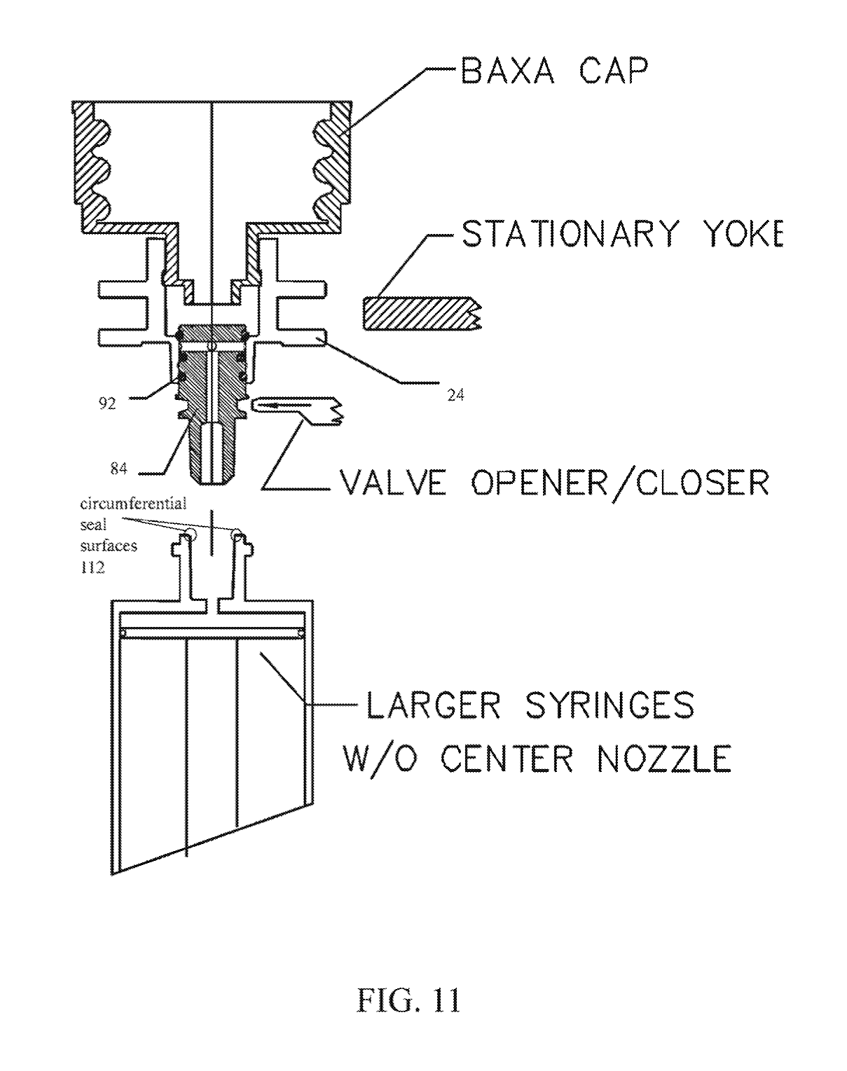

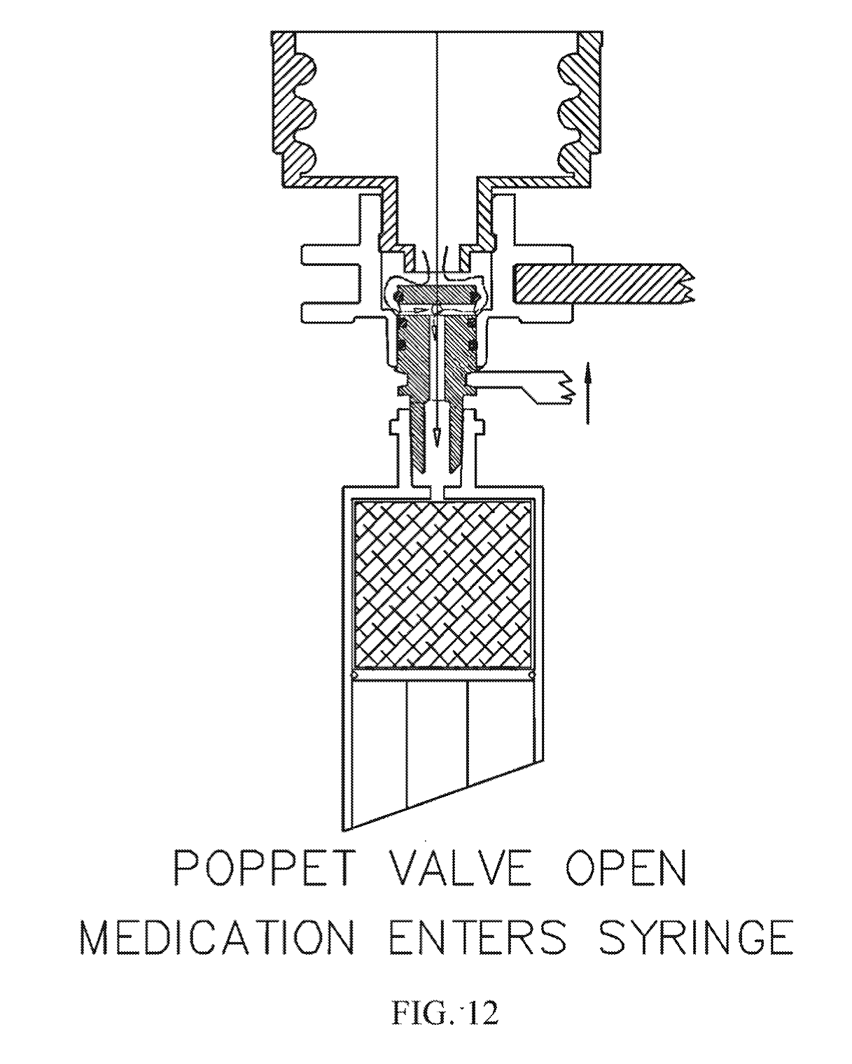

FIGS. 11-12 are sequential perspective views of the push-pull adapter cap 10 of FIGS. 3-9 illustrating how it interfaces with both the larger-size enteral syringe lacking a center nozzle, and the smaller size enteral syringe with center nozzle thereby illustrating the device's ability to adapt to both small syringes(with nozzle) and large syringes (w/o center nozzle). As above, the tip of poppet 84 is externally-tapered and exactly fits the orifice of the enteral syringe female tip preventing any accumulation of medicine in the smaller enteral syringe tip with center nozzle. Thus the tip of the poppet valve is able to be used with both the small enteral syringe with center nozzle and the larger enteral nozzle without the center nozzle.

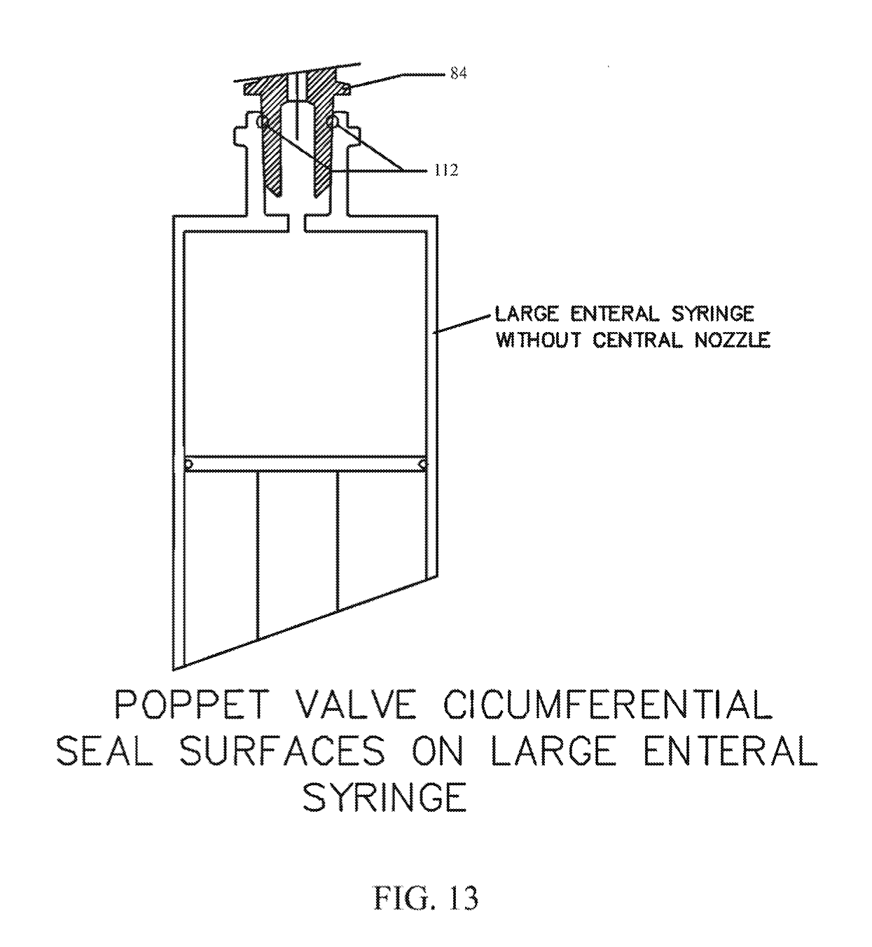

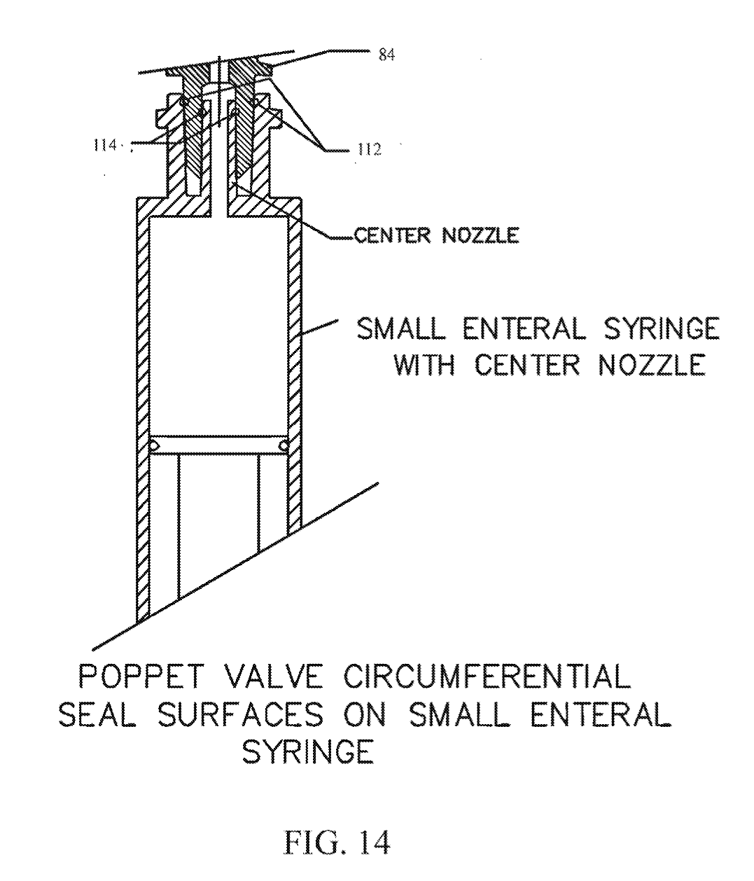

FIG. 13 is a cross-section of the poppet valve 84 of the present invention inserted into a large enteral syringe and illustrating the poppet valve circumferential seal surfaces on the large enteral syringe lacking a center nozzle. As above, the tip of poppet 84 is externally-tapered, i.e., has a tapered outer diameter that exactly mates (“taper-locks”) with the outermost internal surface of the large enteral syringe female tip to create a first circumferential seal 112.

FIG. 14 is a cross-section of the poppet valve 84 of the present invention inserted into a small enteral syringe and illustrating the poppet valve circumferential seal surfaces on the small enteral syringe with internal center nozzle. The poppet valve 84 is configured as above for the first circumferential seal 112 plus the poppet central channel has a tapered outlet configured to mate with the outermost external surface of the enteral syringe nozzle to create a second circumferential seal 114. For the smaller enteral syringes these two seals “taper-lock” both to the outermost internal surface and to the innermost diameter of the internal nozzle of smaller enteral syringes. Despite the type of enteral syringe this configuration completely eliminates unwanted fluid buildup and inaccurate filling volumes.

It should now be apparent that the above design interfaces with and enables opening and closing the flow of medication to enteral syringes with a female tip with or without internal slip-nozzles while the medication container is inverted. As such, the time to load and unload, or upright and invert, the medication container between syringe fillings is eliminated. In addition, the medication container can also be shaken in an inverted position before, during or after a syringe filling operation, when the medication so requires.

One skilled in the art will understand that the push-pull cap 10 may further comprise a 2D barcode to enable easy tracking of the medication container bearing that cap. Each barcode (or possibly RFID tag or other label) preferably references the following information:
Batch number
Expiry date
Storage instructions
Product name
Strength
Name of the active ingredient(s)
Dose form
Warning statements
NDC number (National Drug Code)
Does product need to be shaken before use? If so, how often?
Does product need to be refrigerated before use? If so, temp?
Does product need to be protected from light?
Volume of original bulk medication container?

In addition, molded surface features or textures may be provided on the outer surface of each cap to provide a gripping surface.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

We claim:

1. A push-pull valve for use with a medication container adapter for filling enteral syringes with medication from the medication container when the medication container is in an inverted positon on a syringe filler, comprising:
a stationary portion for attachment to the medicine container adapter, said stationary portion comprising an annular member having a central channel; and
a poppet slidably inserted into the stationary portion, said poppet comprising an annular member having a central channel, a radial inlet to said central channel, and a fluted axial outlet from said central channel.

2. The valve according to claim 1, wherein said poppet further comprises a radial inlet to said central channel, and a fluted axial outlet from said central channel.

3. The valve according to claim 1, attached to a medication container adapter.

4. The valve according to claim 3, further comprising at least one annular flange protruding from said stationary portion for manipulation by said filling system.

5. The valve according to claim 4, further comprising a pair of spaced annular flanges protruding from said stationary portion for manipulation by said filling system.

6. The valve according to claim 1, further comprising an annular shoulder encircling said stationary portion for press-fit insertion onto a medicine container adapter cap.

7. The valve according to claim 1, further comprising at least one O-ring about said poppet.

8. The valve according to claim 1, further comprising at least one O-ring seat formed as an annular recess in said stationary portion.

9. A medication container adapter with integrated push-pull valve as described in claim 3, configured to screw onto a medication container.

10. A medication container adapter with integrated push-pull valve as described in claim 3, wherein said stationary portion comprises a plurality of inwardly-protruding annular resilient ribs for a fluid seal.

11. A medication container adapter with integrated push-pull valve as described in claim 3, wherein said stationary portion comprises a plurality of outwardly protruding annular resilient ribs for a fluid seal.

12. A medication container adapter with integrated push-pull valve as described in claim 3, configured press-fit insertion onto a medication container neck.

13. A medication container adapter with integrated push-pull valve as described in claim 3, wherein said push-pull valve comprises a poppet having a radial inlet to said central channel, and a fluted axial outlet from said central channel.

14. The medication container adapter with integrated push-pull valve as described in claim 3, further comprising at least one annular flange protruding from said stationary portion for manipulation by said filling system.

15. The medication container adapter with integrated push-pull valve as described in claim 14, further comprising a pair of spaced annular flanges protruding from said stationary portion for manipulation by said filling system.

16. The valve according to claim 1, wherein said poppet has a tip configured to seal along an internal circumference of the enteral syringe tip, and the fluted axial outlet of said poppet is configured to seal along an outer circumference of the center nozzle within the enteral syringe tip.

17. The valve according to claim 1, wherein said poppet has a tapered outer diameter that mates with the internal, outermost, circumference of the enteral syringe tip creating a seal.

18. A valve according to claim 17, in like manner the internal diameter of the poppet, taper-locks to the outermost diameter of the internal nozzle of the enteral syringe tip.

19. An adapter cap for attachment to a medicine container to allow attachment of an enteral syringe for filling the syringe with medication when the medicine container is in an inverted positon in a filling system, comprising:
a stationary portion comprising an annular member having a central channel; and
a movable push-pull valve in the stationary portion.

20. For use in a hospital filling system, an adapter cap for attachment to a medicine container to allow attachment of a syringe, said adapter cap comprising:
an annular member configured for attachment to a medicine container, said annular member having a central channel; and
a poppet slidably inserted into the annular member, said poppet comprising a central channel.

21. The adapter cap according to claim 20, wherein said poppet further comprises a radial inlet to said central channel, and a fluted axial outlet from said central channel.

22. The adapter cap according to claim 20, further comprising at least one annular flange protruding from said annular member for manipulation by said filling system.

23. The adapter cap according to claim 21, further comprising a pair of spaced annular flanges.

24. The adapter cap according to claim 21, further comprising an annular flange about said poppet for push-pull operation.

25. The adapter cap according to claim 20, further comprising an annular shoulder encircling said stationary portion for press-fit insertion onto said medicine container adapter cap.

26. The adapter cap according to claim 20, further comprising screw-threads for attachment to a medication container neck.

27. The adapter cap according to claim 20, configured to be press fitted into the neck of the medication container.

28. The adapter cap according to claim 20, further comprising a plurality of resilient ribs external to said stationary portion for forming a fluid seal about a neck of said medicine container.

29. The adapter cap according to claim 20, further comprising a plurality of resilient ribs internal to said stationary portion for forming a fluid seal inside a neck of said medicine container cap.

30. The adapter cap according to claim 20, further comprising at least one 0-ring about said poppet.

31. The adapter cap according to claim 30, further comprising at least one 0-ring seat formed as an annular recess in said annular member.

32. A method of using an adapter cap to attach an enteral syringe to a medicine container for filling the syringe with medication, comprising the steps of:
removing a manufacturer-supplied medicine container cap from a medicine container;
installing an adapter cap on said medicine container, said adapter cap having a stationary portion comprising an annular member having a central channel, and a push-pull valve in the movable portion;
inverting the medication container;
attaching an enteral syringe for filling the syringe with medication from said medicine container;
opening said adapter cap push-pull valve;
withdrawing a plunger of said syringe to thereby fill said syringe with medicine from said medicine container;
closing the said adapter cap push-pull valve; and
withdrawing the syringe from the medication container.

33. For use in a system for automated filling of syringes with a selected bulk medicine container, an adapter cap having an annular body defined by an aperture through said body, and a push-pull valve slidably inserted in said annular body.

34. The adapter cap according to claim 32, wherein said annular body is configured for screw-attachment onto a neck of said medicine container.

35. The adapter cap according to claim 32, wherein said annular body is configured for press-fit insertion onto a neck of said medicine container.
